(19) United States
(12) Patent Application Publication
(10) Pub. No.: US 2025/0312195 A1
(43) Pub. Date: Oct. 9, 2025

(54) PROVIDING A DEPTH OVERLAY FOR AN OPHTHALMIC SYSTEM

(71) Applicant: Alcon Inc., Fribourg (CH)

(72) Inventors: Mario Abraham, Burgthann (DE); Martin Gruendig, Rangsdorf (DE)

(21) Appl. No.: 19/087,165

(22) Filed: Mar. 21, 2025

Related U.S. Application Data
(60) Provisional application No. 63/631,509, filed on Apr. 9, 2024.

(51) Int. Cl. A61F 9/008; G06T 7/246; G06T 7/593; G06T 7/73; G06T 19/00

(52) U.S. Cl. CPC A61F 9/008; G06T 7/248; G06T 7/593; G06T 7/74; G06T 19/00; A61F 2009/00846; A61F 2009/00897; G06T 2207/10012; G06T 2207/30041

(57) ABSTRACT

In certain embodiments, an ophthalmic system for tracking and imaging an eye of a patient includes a tracking camera, a stereoscopic camera, and a computer. The tracking camera tracks a patient feature of the patient. The stereoscope camera system generates left image data and right image data of the eye and the patient feature to yield a stereoscopic image of the eye and the patient feature. The computer generates a depth overlay representing the patient feature at a z-location relative to a z-axis of a system coordinate system. The depth overlay includes a left depth overlay and a right depth overlay. The computer inserts the left depth overlay into the left image data and the right depth overlay into right image data to yield the stereoscopic image of the eye with the depth overlay representing the patient feature at the z-location.

The present disclosure relates generally to ophthalmic systems, and more particularly to providing a depth overlay for an image displayed by an ophthalmic system.

### BACKGROUND

In ophthalmic laser surgery, the eye should be at the correct distance from the laser system to properly treat the eye. Surgical systems aim the focal point of the laser beam at specific locations of the eye in order to treat the eye. If the eye is not at the proper distance, the laser beam might not effectively treat the eye and may even damage the eye. According to some techniques, laser diodes that yield reflections on the eye may be used to check the distance. When the eye is at the correct distance, the reflections form a specific pattern on the eye.

### BRIEF SUMMARY

In certain embodiments, an ophthalmic system for tracking and imaging an eye of a patient includes a tracking camera, a stereoscopic camera, and a computer. The tracking camera tracks a patient feature of the patient. The stereoscope camera system generates left image data and right image data of the eye and the patient feature to yield a stereoscopic image of the eye and the patient feature. The computer generates a depth overlay representing the patient feature at a z-location relative to a z-axis of a system coordinate system. The depth overlay includes a left depth overlay and a right depth overlay. The computer inserts the left depth overlay into the left image data and the right depth overlay into right image data to yield the stereoscopic image of the eye with the depth overlay representing the patient feature at the z-location.

Embodiments may include none, one, some, or all of the following features:
The computer determines the z-location of the depth overlay according to a reference plane that is a treatment plane or a diagnostic plane.
The computer determines the z-location of the depth overlay according to the distance of the patient feature from a reference plane.
The patient feature is the pupil of the eye. The computer determines the z-location of the depth overlay according to the anterior chamber depth of the eye.
The computer determines the z-location of the depth overlay according to a dimension of the eye, the dimension comprising a length selected the following: the anterior chamber depth, axial length, limbal diameter, or rotation center of the eye.
The computer generates a real-time overlay representing the patient feature shown in the stereoscopic image and inserts the real-time overlay into the left image data and the right image data to yield the stereoscopic image of the eye with the real-time overlay representing the patient feature at the z-location shown in the stereoscopic image.
The computer generates a real-time overlay representing the patient feature shown in the stereoscopic image. The real-time overlay includes a left real-time overlay and a right real-time overlay. The computer inserts the left real-time overlay into the left image data and the right real-time overlay into the right image data to yield the stereoscopic image of the eye with the real-time overlay representing the patient feature at the z-location shown in the stereoscopic image. The computer may detect that the real-time overlay is aligned with the depth overlay and provide a notification that the real-time overlay is aligned with the depth overlay. The computer may change one or more graphical features of the real-time overlay in response to detecting that the real-time overlay is aligned with the depth overlay and/or in response to detecting that the real-time overlay is moving closer to alignment with the depth overlay.
The computer performs image processing to identify the patient feature shown in the stereoscopic image of the eye and calculates an adjustment to a relative distance between the eye and the system to align the patient feature with the depth overlay. The computer may: provide a description of the adjustment to a user; detect that the adjustment has been performed to align the patient feature with the depth overlay and provide a notification to a user that the patient feature is aligned with the depth overlay; or automatically perform the adjustment to align the patient feature with the depth overlay.
The computer: performs image processing to identify the patient feature shown in the stereoscopic image of the eye; detects that the patient feature is not aligned with the depth overlay; and provides a notification that the eye is not at the z-location.
The system further includes a display device that presents the stereoscopic image to a viewer by presenting the left image data to the left eye of the viewer and presenting the right image data to the right eye of the viewer. The display device may be oculars that presents the left image data to the left ocular of the oculars and presents the right image data to the right ocular of the oculars. The display device may be a headset that presents the left image data to the left screen of the headset and presents the right image data to the right screen of the headset. The display device may be a display that presents the left image data polarized for the left eye of the viewer and presents the right image data polarized for the right eye of the viewer. The display device may be a display that presents the left image data angled for the left eye of the viewer and presents the right image data angled for the right eye of the viewer.
The patient feature is the pupil of the eye, and the depth overlay is substantially the same size as the pupil.
The patient feature is the limbus of the eye, and the depth overlay is substantially the same size as the limbus.
The patient feature is the eyeball of the eye, and the depth overlay is substantially the same size as the eyeball.
The computer detects that the patient feature is aligned with the depth overlay and provides a notification that the patient feature is aligned with the depth overlay.
The computer detects that the patient feature is aligned with the depth overlay and changes one or more graphical features of the depth overlay in response to detecting that the patient feature is aligned with the depth overlay.
The computer detects that the patient feature is moving closer to alignment with the depth overlay and changes one or more graphical features of the depth overlay in response to detecting that the patient feature is moving closer to alignment with the depth overlay.

In certain embodiments, a method for tracking and imaging an eye of a patient includes tracking a patient feature of the patient, generating left image data and right image data of the eye and the patient feature to yield a stereoscopic image of the eye and the patient feature, and generating, by a computer, a depth overlay representing the patient feature at a z-location relative to a z-axis of a system coordinate system. The depth overlay includes a left depth overlay and a right depth overlay. The method includes inserting, by the computer, the left depth overlay into the left image data and the right depth overlay into right image data to yield the stereoscopic image of the eye with the depth overlay representing the patient feature at the z-location.

Embodiments may include none, one, some, or all of the following features:
The patient feature is the pupil of the eye. The computer determines the z-location of the depth overlay according to the anterior chamber depth of the eye.
The method further includes: generating a real-time overlay representing the patient feature shown in the stereoscopic image; and inserting the real-time overlay into the left image data and the right image data to yield the stereoscopic image of the eye with the real-time overlay representing the patient feature at the z-location shown in the stereoscopic image.

### BRIEF DESCRIPTION OF THE DRAWINGS

FIG. 1 illustrates an example of an ophthalmic system for tracking and imaging an eye of a patient, according to certain embodiments;

FIG. 2 illustrates an example of calculating the z-location of a depth overlay, according to certain embodiments;

FIGS. 3A and 3B illustrate an example of a depth overlay and a real-time overlay in a three-dimensional (3D) image of the eye, according to certain embodiments;

FIGS. 4A and 4B illustrate examples of a depth overlay and a real-time overlay, according to certain embodiments;

FIGS. 5A to 5C illustrate examples of a depth overlay and a real-time overlay for the pupil of an eye, according to certain embodiments;

FIGS. 6A to 6C illustrate examples of a depth overlay and a real-time overlay for the limbus of an eye, according to certain embodiments;

FIGS. 7A to 7C illustrate an example of a depth overlay for the eyeball of an eye, according to certain embodiments; and

FIG. 8 illustrates an example of a method for providing a depth overlay for a 3D image that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Known techniques for monitoring the distance between the patient's eye and a laser surgical system have issues. Laser diodes that yield a particular pattern on the eye disturb the surgeon's view of the eye. Accordingly, the diodes are typically deactivated after the eye has been aligned, making height control during surgery difficult.

Certain embodiments of the system described herein provide a graphical overlay on a real-time image of an eye that indicates the appropriate distance of the eye from the system. In an example system, a tracking camera tracks the eye, and a stereoscopic camera system yields a real-time stereoscopic image of the eye. A computer generates an overlay that indicates the distance where the eye should be when it is properly aligned with the system and inserts the overlay into the stereoscopic image of the eye. In some situations, the surgeon may align the eye using the overlay as a guide. In other situations, the system may automatically align the eye.

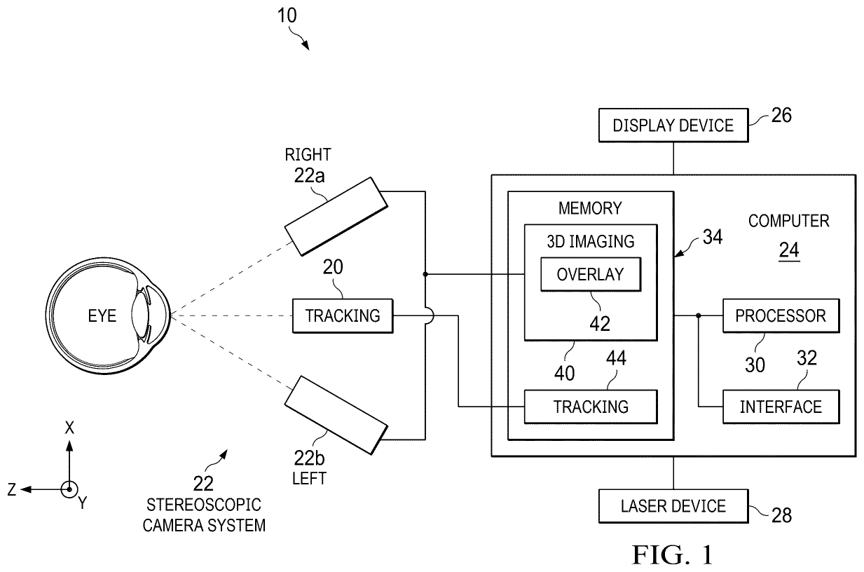

FIG. 1 illustrates an example of an ophthalmic system 10 for tracking and imaging an eye of a patient, according to certain embodiments. In the example, system 10 includes a tracking camera 20, stereoscopic camera system 22 (with stereoscopic cameras 22a and 22b), computer 24, display device 26, and laser device 28, coupled as shown. Computer 24 includes a processor 30, an interface (IF) 32, and a memory 34, coupled as shown. Memory 34 stores applications such as a three-dimensional (3D) imaging application 40 (which generates an overlay 42) and a tracking application 44.

For case of explanation, the embodiments are described using the following example xyz-coordinate system, which may be regarded as the coordinate system of system 10, although any suitable coordinate system may be used. In the example, the z-axis is aligned with the optical axis of tracking camera 20, and the xy-plane is orthogonal to the z-axis. Tracking camera 20 and stereoscopic camera system 22 may each have their own sub-coordinate system that can be translated to the common coordinate system of system 10. In addition, the position of an object may refer to the location and/or orientation of the object.

As an example overview, tracking camera 20 tracks a patient feature of the patient (e.g., the pupil) in the xy-plane. Stereoscopic cameras 22 generate left image data and right image data that can yield a three-dimensional (3D) stereoscopic image of the eye showing the patient feature. Computer 24 generates a depth overlay 42 that represents the z-location (as well as the xy-location) of the patient feature when the eye is at the target distance, i.e., the proper distance from the system for, e.g., a surgical or diagnostic procedure. The user can align the eye by checking whether the actual feature in the real-time image coincides with the feature in the overlay 42. Depth overlay 42 includes a left depth overlay and a right depth overlay. Computer 24 inserts the left depth overlay into the left image data and the right depth overlay into right image data to yield the stereoscopic image of the eye with the depth overlay representing the target z-location of the patient feature.

Tracking camera 20 tracks a feature of the patient, e.g., the pupil, and provides the xy-position of the feature to computer 24. Tracking camera 20 may be any suitable camera, such as an infrared or visible light camera that operates at any suitable refresh rate, e.g., 100 to 250, 250 to 500, 500 to 1000, or greater than 1000 Hz. Tracking camera 20 may be located at any suitable position, e.g., on-axis with the patient's eye such that the optical axis of camera 20 is aligned with an axis (e.g., optical or visual) of the patient's eye. Any suitable patient feature may be tracked, e.g., the pupil, limbus, iris, eye contour, eyeball, upper/lower lid, eyebrow, nose, or other eye or facial feature.

Stereoscopic camera system 22 generates image data to yield a real-time 3D image. Stereoscopic camera system 22 includes lenses with a separate image sensor for each lens, which allows system 22 to simulate human binocular vision and capture 3D images in stereo photography. In the example, cameras 22a and 22b are arranged off-axis to yield a 3D image. The distance between the sensors is known, so the depth of an object in the 3D image can be determined.

Computer 24 sends instructions to the components of system 10 (e.g., tracking camera 20, stereoscopic camera system 22, display device 26, and/or laser device 28) to generate a 3D image of the patient's eye and provide an overlay for the image. In certain embodiments computer 24 uses 3D imaging application 40 to generate depth overlay 42 that indicates the z-location of the patient feature when the eye is at the specific distance from the system and to insert overlay 42 into the 3D image. The user can use the overlay to align the eye in the z-direction.

In certain embodiments, 3D imaging application 40 generates a real-time overlay representing the actual position of the patient feature. In the embodiments, application 40 detects the feature in the right and left image data. Application 40 superimposes a left real-time overlay onto the feature in the left image data and a right real-time overlay onto the feature of the right image data to yield the real-time stereoscopic image of the eye with the real-time overlay superimposed onto the feature.

Depth overlay 42 may be designed to indicate any suitable distance from the system. In certain situations, overlay 42 may indicate the z-location of a patient feature (such as an eye feature) when the eye is at the optimal z-location for a treatment procedure, such as a laser surgical procedure. In other situations, overlay 42 may indicate the z-location of the feature when the eye is at the optimal z-location for a diagnostic procedure, such as an imaging procedure. Depth overlay 42 may represent any suitable patient feature, such as the pupil, iris, sclera, or other facial or eye feature.

Display device 26 presents left and right image data to the left and right eyes, respectively, of a viewer to create a stereoscopic image for the viewer. Examples include oculars, headsets, and display screens or monitors. In certain embodiments, display device 26 includes oculars that present the left image data in the left ocular and the right image data in the right ocular to create the stereoscopic image. In certain embodiments, display device 26 includes a headset that present the left image data on the left screen of the headset and the right image data on the right screen. In certain embodiments, display device 26 includes a 3D screen that directs the left image data to the left eye and the right image data to the right eye, by, e.g., polarization or angles. For example, the left image data may have a polarization that passes through the left lens of the 3D glasses of a viewer, and the right image data may have a polarization that passes through the right lens of the 3D glasses. As another example, the left image data may be directed at an angle that points towards the left eye of the viewer, and the right image data may be directed at an angle that points towards the right eye.

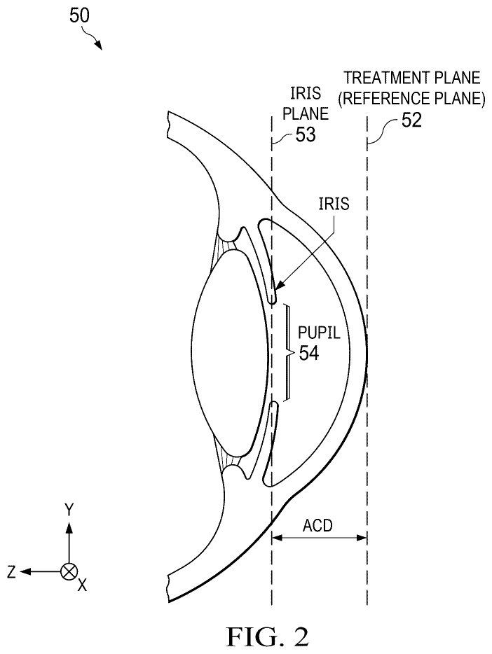

FIG. 2 illustrates an example 50 of calculating the z-location of a depth overlay, according to certain embodiments. Computer 24 calculates the z-location according to what the depth overlay is designed to represent and/or the type of patient feature. The depth overlay may be designed to represent the target position of a patient feature (e.g., pupil 54) relative to a reference plane, such as a treatment or diagnostic plane of a procedure. The distance between the target position and the reference plane may be determined from measurements of the eye, e.g., average measurements of the patient's cohort (e.g., a group of similarly situated patients, such as those with a similar age, similar gender, similar eye size, similar prognosis, similar eye prescription, etc., or any combinations thereof) and/or actual measurements of the patient's eye.

In example 50, the depth overlay is designed to show the z-location of pupil 54 when the eye is aligned in the z-direction relative to the reference plane, which is the treatment plane 52 for a laser surgical procedure at the corneal surface. The eye feature is the pupil 54 defined by the iris located in the iris plane 53. The distance between the eye feature and reference plane can be determined from, e.g., the anterior chamber depth (ACD). Accordingly, pupil 54 should appear in the depth overlay at a distance equivalent to the ACD beyond treatment plane 52 towards the posterior of the eye. In other examples, the distance may be determined from other ocular biometry measurements, e.g., axial length, limbal diameter, and/or rotation center of the eye.

Referring back to FIG. 1, computer 24 may perform additional functions to assist with aligning the eye. In certain embodiments, computer 24 identifies the actual patient feature in the real-time stereoscopic image of the eye and determines the z-location of the actual feature. For example, the actual z-location may be calculated using the distance between the sensors of the stereoscopic cameras 22. From the difference between the actual z-location and the z-location of the feature given by the overlay, computer 24 may then calculate an adjustment that aligns the actual feature with the overlay.

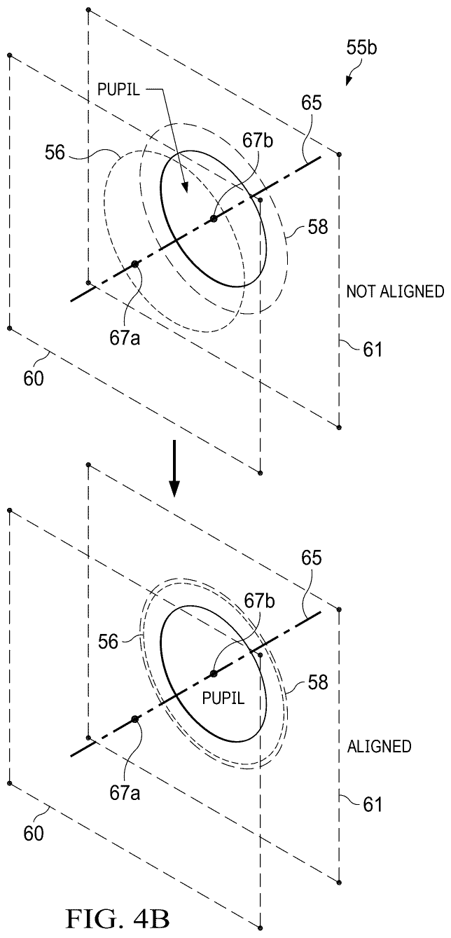

In certain embodiments, computer 24 may provide a description of the adjustment to the user. The notification may have any suitable form, e.g., a message or graphical element indicating the patient's eye should be moved a specific distance and/or in a particular direction. An example of such notification is described with reference to FIGS. 4A and 4B. Computer 24 may detect that the adjustment has been performed to align the patient feature with the depth overlay and then provide notification that the patient feature is aligned. In certain embodiments, computer 24 may automatically perform the adjustment.

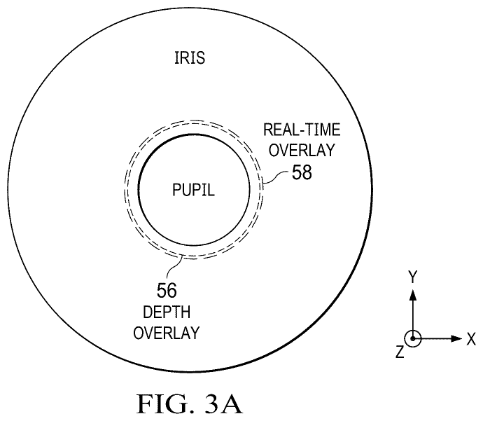

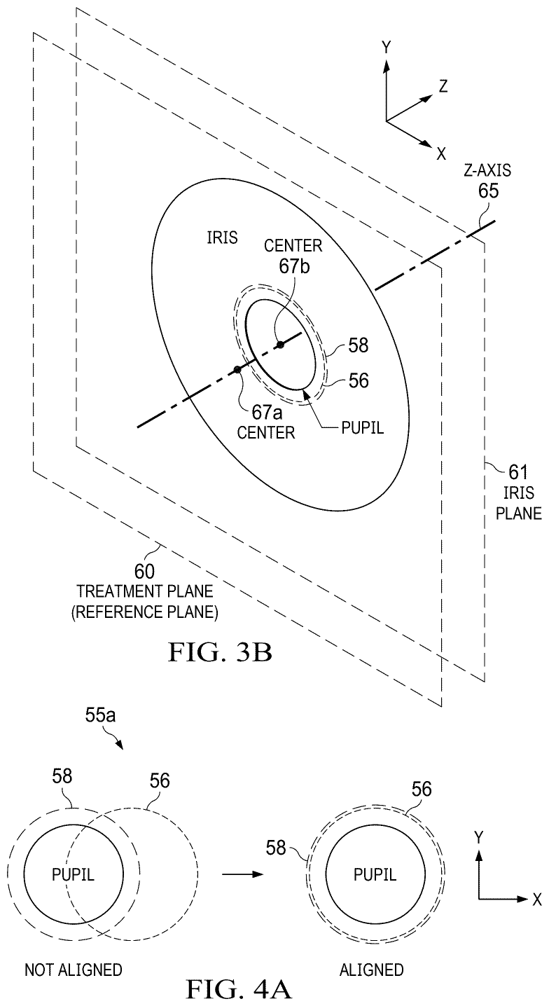

FIGS. 3A and 3B illustrate an example of a depth overlay 56 and a real-time overlay 58 in a 3D image of the eye (which may be referred to collectively as overlays), according to certain embodiments. In certain embodiments, system 10 presents depth overlay 56 that indicates the target position of an eye feature. The eye feature can be aligned with depth overlay 56 to align the eye. In other embodiments, as shown in FIGS. 3A and 3B, system 10 also presents real-time overlay 58 that indicates the actual position of the eye feature. Real-time overlay 58 can facilitate aligning the eye feature with depth overlay 56 to align the eye.

An overlay may be a graphical element having any suitable graphical features, such as any suitable shape, size, color, or line pattern (e.g., solid, dashed, dash-dotted, or dotted line). In certain embodiments, an overlay may be a size and/or shape similar to those of the feature of the eye (the “eye feature”) the overlay is representing, e.g., a pupil or iris overlay may be circular or elliptical or an eyeball overlay may be spherical. In the embodiments, the size and/or shape of the overlay may be determined from the size and/or shape of an average eye feature, e.g., the average eye feature of the patient's cohort. In other embodiments, the size and/or shape of the depth overlay 56 and/or real-time overlay of the eyeball may be determined from measurements of the patient's eye. In certain embodiments, an overlay may include elements indicating a geometric detail of the feature, e.g., marking(s) that indicate the center and/or boundary of a pupil or iris. In certain embodiments, an overlay may be a color that contrasts with the real-time image, e.g., if the real-time image is in black-and-white, the overlay may be a primary color. In certain embodiments, overlays 56 and/or 58 appear as augmented reality graphical elements on the real-time image of the eye.

Depth overlay 56 and real-time overlay 58 may be visually distinguished from each other in any suitable manner. In the example, overlays 56 and 58 are graphically distinguished by size, e.g., overlay 58 is slightly larger than overlay 56. The eye is aligned when depth overlay 56 is concentric with real-time overlay 58. However, overlays 56 and 58 may be graphically distinguished by any suitable graphical feature, e.g., by shape, size, color, or line pattern.

System 10 may provide a notification that the eye is in alignment. The notification may be an audio, visual, haptic, and/or other sensory cue. The cue may increase or decrease (e.g., in intensity, rate, or volume) based on proximity to alignment. In some embodiments, the system 10 may cause a visual change in the real-time overlay 58 and/or the depth overlay 56 when the eye is in alignment, such as change a graphical feature of the real-time overlay 58 and/or the depth overlay 56. For example, the real-time overlay 58 may change color, such as changing color to match the color of the depth overlay 56, in response to the real-time overlay 58 being aligned with the depth overlay 56. As another example, the real-time overlay 58 may change line pattern, such as changing line pattern to match the line pattern of the depth overlay 56, in response to the real-time overlay 58 being aligned with the depth overlay 56. It will be appreciated that any other visual change may be applied to either or both of the overlays 56 and/or 58 to visually convey the proper alignment of the eye.

In some embodiments, other cues may indicate when the eye is aligned. For example, an audio tone may be played when the eye is aligned. As another example, a vibration or other haptic feedback may be experienced by the user when the eye is aligned. As a further example, a sequence of audio tones or beeps may be played with increasing or decreasing tempo based on how close to alignment the real-time overlay 58 is with the depth overlay 56.

FIG. 3A is an en face view of depth overlay 56 and real-time overlay 58 in the xy-plane of the eye feature plane, e.g., the iris plane. FIG. 3B is a perspectival view of depth overlay 56 and real-time overlay 58. In the example, depth overlay 56 represents the target position of the pupil (located at the iris plane 61) relative to the reference plane, which is the treatment plane 60 in this example. The z-axis 65 passes through the centers 67a and 67b of the planes 60 and 61, respectively. In the figure, real-time overlay 58 is concentrically aligned with depth overlay 56 at the iris plane 61, indicating that the eye is properly aligned. In this example, the reference plane, i.e., the treatment plane 60, is anterior to depth overlay 56 and real-time overlay 58. In other examples, a different reference plane may be selected, such as a lens plane that is posterior to depth overlay 56 and real-time overlay 58.

FIGS. 4A and 4B illustrate examples 55 (55a and 55b, respectively) of depth overlay 56 and real-time overlay 58, according to certain embodiments. FIG. 4A illustrates an example 55a with overlays 56 and 58 indicating an eye that is not aligned and then aligned in the xy-plane. In the example, depth overlay 56 that is not superimposed over real-time overlay 58 indicates that the eye is not aligned with the depth overlay, and depth overlay 56 superimposed over real-time overlay 58 indicates that the eye is aligned with the depth overlay.

FIG. 4B illustrates an example 55b with overlays 56 and 58 indicating an eye aligned in the xy-directions but not z-direction and then aligned in the xyz-directions. In the example, depth overlay 56 is located in a plane between treatment plane 60 and iris plane 61, indicating the eye is not at the target z-position. When depth overlay 56 is located in iris plane 61, the eye is at the target z-position.

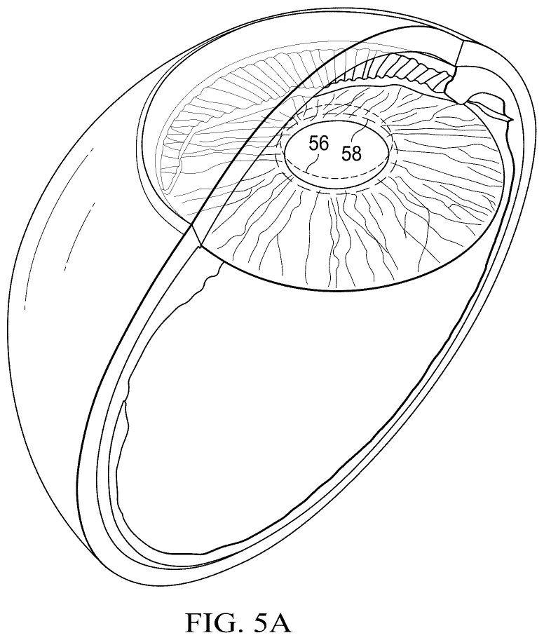

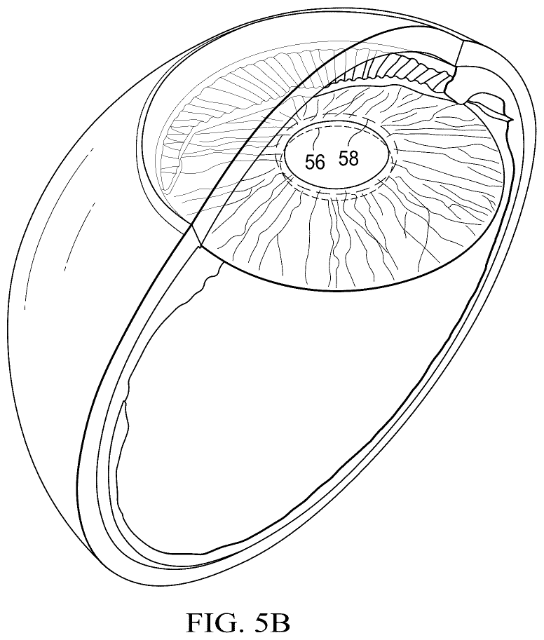

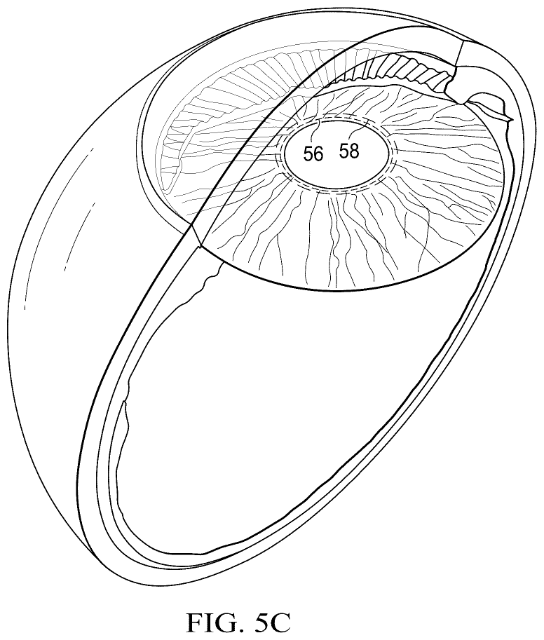

FIGS. 5A to 5C illustrate examples of depth overlay 56 and real-time overlay 58 for the pupil of an eye, according to certain embodiments. Depth overlay 56 indicates the target position of the pupil, and real-time overlay 58 indicates the actual position of the pupil. In the examples, overlays 56 and 58 are substantially the same shape as the pupil and are graphically distinguished from each other by size (overlay 58 is slightly larger than overlay 56) and by line pattern. Additionally or alternatively, the overlays 56 and 58 may be distinguished from each other by color. FIG. 5A shows the eye posterior to the treatment plane as depth overlay 56 is anterior to the pupil. FIG. 5B shows the eye anterior to the treatment plane as depth overlay 56 is posterior to the pupil. FIG. 5C shows the eye properly aligned in the z-direction.

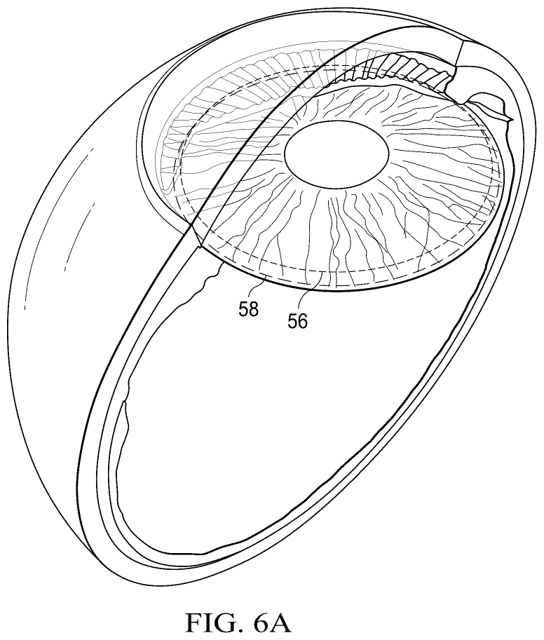

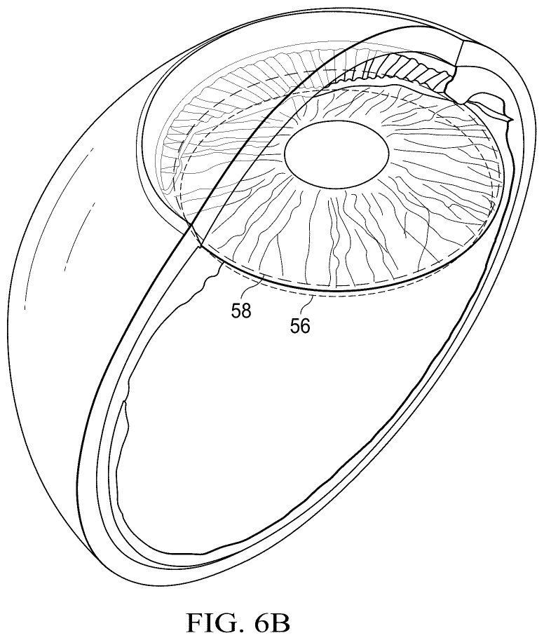

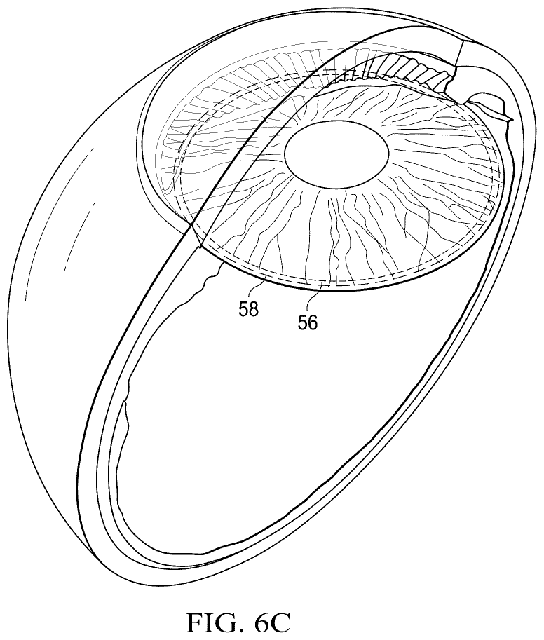

FIGS. 6A to 6C illustrate examples of depth overlay 56 and real-time overlay 58 for the limbus of an eye, according to certain embodiments. Depth overlay 56 indicates the target position of the limbus, and real-time overlay 58 indicates the actual position of the limbus. In the examples, overlays 56 and 58 are substantially the same shape as the limbus and are graphically distinguished from each other by size (overlay 58 is slightly larger than overlay 56) and by line pattern. FIG. 6A shows the eye posterior to the treatment plane as depth overlay 56 is anterior to the limbus. FIG. 6B shows the eye anterior to the treatment plane as depth overlay 56 is posterior to the limbus. FIG. 6C shows the eye properly aligned in the z-direction.

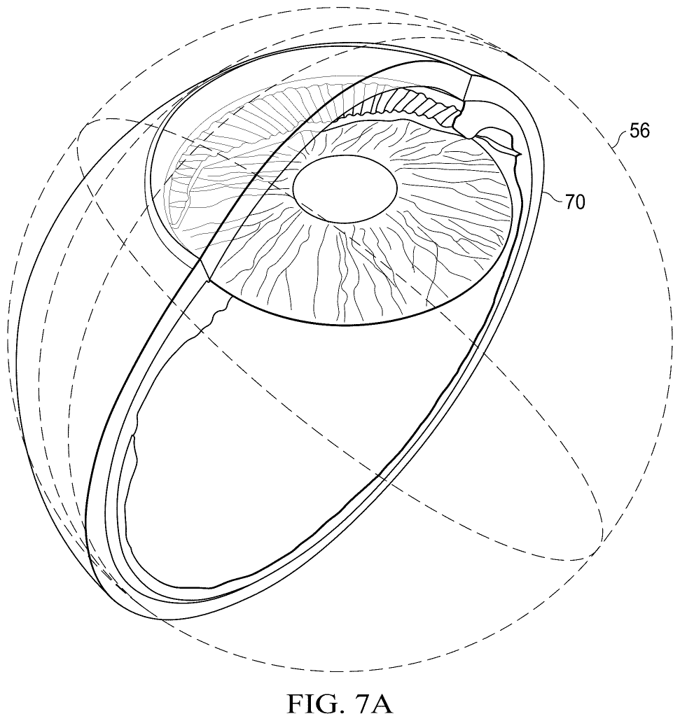

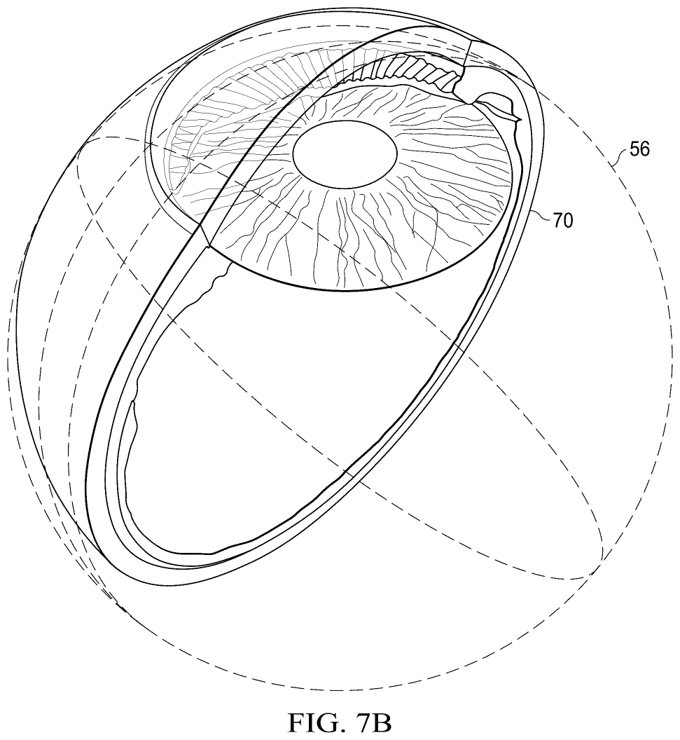

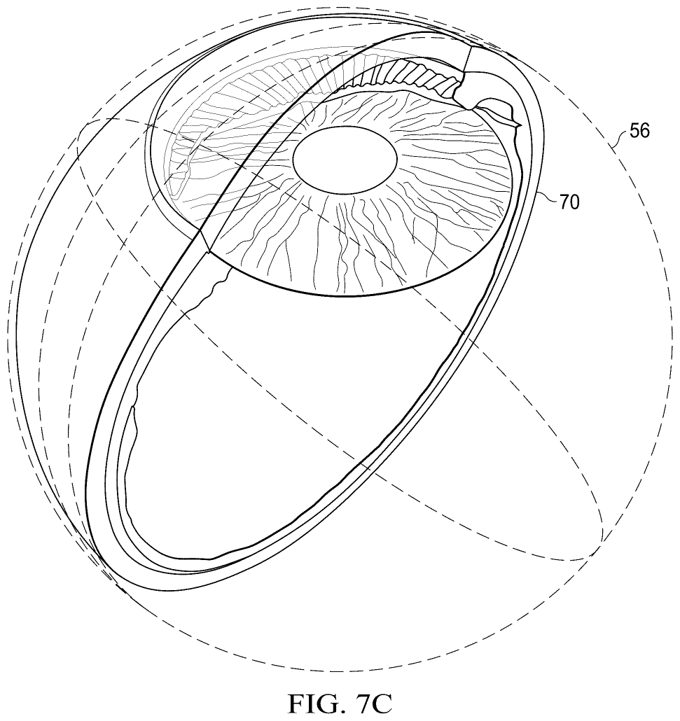

FIGS. 7A to 7C illustrate examples of depth overlay 56 for the eyeball 70 of an eye, according to certain embodiments. Depth overlay 56 indicates the target position of the eyeball with one or more lines (e.g., one or more longitudinal lines and/or one or more latitudinal lines) that outline the eyeball. In some embodiments, rather than using an overlay of the pupil or the limbus to align the eye, an overlay of the eyeball itself may be used to facilitate alignment. In these and other embodiments, the depth overlay 56 may indicate the target position of the eyeball 70 of the eye. Although not shown in the figure, system 10 may also display a real-time overlay 58 that indicates the actual position of the eyeball. The real-time overlay may have one or more lines (e.g., one or more longitudinal lines and/or one or more latitudinal lines) that outline the eyeball.

The depth overlay 56 and/or real-time overlay of the eyeball may have any suitable appearance. In certain embodiments, the size and/or shape of the depth overlay 56 and/or real-time overlay of the eyeball may be determined from the size and/or shape of an average eyeball, e.g., the average eyeball of the patient's cohort. In other embodiments, the size and/or shape of the depth overlay 56 and/or real-time overlay of the eyeball may be determined from measurements of the patient's eye.

FIG. 7A shows the eye posterior to the treatment plane as depth overlay 56 is anterior to the eyeball. FIG. 7B shows the eye anterior to the treatment plane as depth overlay 56 is posterior to the limbus. FIG. 7C shows the eye properly aligned in the z-direction.

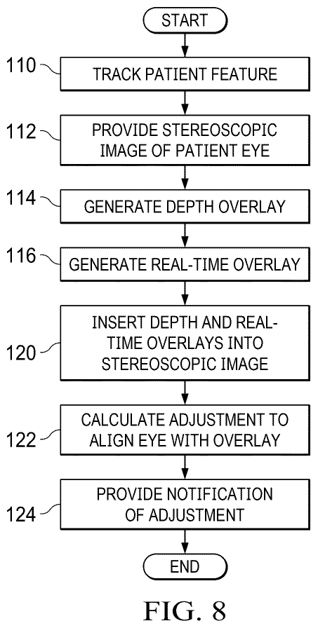

FIG. 8 illustrates an example of a method for providing a depth overlay for a 3D image that may be performed by system 10 of FIG. 1, according to certain embodiments. The method starts at step 110, where tracking camera 20 tracks a patient feature in the xy-plane of the coordinate system of system 10. Stereoscope camera system 22 provides a stereoscopic image at step 112 showing the patient feature. Camera system 22 may generate left and right image data that yield the stereoscopic image.

Computer 24 generates depth overlay 56 at step 114 and generates real-time overlay 58 at step 116. The z-location of the patient feature in the depth overlay may be determined according to a reference plane (e.g., a treatment or diagnostic plane) and may indicate the distance between the patient feature and reference plane. Computer 24 inserts depth overlay 56 and real-time overlay 58 into the stereoscopic image at step 120. For example, computer 24 inserts the left depth overlay into the left image data and the right depth overlay into right image data to yield the stereoscopic image of the eye with the depth overlay. Computer 24 also inserts the real-time overlay into the left and right image data to yield the stereoscopic image of the eye with the real-time overlay representing the actual patient feature.

Computer 24 calculates an adjustment between the eye and system 10 at step 122 that aligns the patient feature with the depth overlay. For example, computer 24 identifies the patient feature in the stereoscopic image and calculates the adjustment of the relative distance between the eye and system 10 that aligns the feature. In certain embodiments, system 10 provides a description of the adjustment to the user. In the embodiments, computer 24 may detect that the adjustment has been performed and provide a notification that the feature is aligned. In certain embodiments, system 10 automatically performs the adjustment to align the patient feature.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic system for tracking and imaging an eye of a patient, comprising:
a tracking camera configured to track a patient feature of the patient;
a stereoscope camera system configured to generate left image data and right image data of the eye and the patient feature to yield a stereoscopic image of the eye and the patient feature; and
a computer configured to:
generate a depth overlay representing the patient feature at a z-location relative to a z-axis of a system coordinate system, the depth overlay comprising a left depth overlay and a right depth overlay; and
insert the left depth overlay into the left image data and the right depth overlay into right image data to yield the stereoscopic image of the eye with the depth overlay representing the patient feature at the z-location.

2. The system of claim 1, the computer configured to:
determine the z-location of the depth overlay according to a reference plane comprising a treatment plane or a diagnostic plane.

3. The system of claim 1, the computer configured to:
determine the z-location of the depth overlay according to a distance of the patient feature from a reference plane.

4. The system of claim 1, wherein:
the patient feature comprises a pupil of the eye; and
the computer is configured to determine the z-location of the depth overlay according to an anterior chamber depth of the eye.

5. The system of claim 1, the computer configured to determine the z-location of the depth overlay according to a dimension of the eye, the dimension comprising a length selected the following: an anterior chamber depth, an axial length, a limbal diameter, or a rotation center of the eye.

6. The system of claim 1, the computer configured to:
generate a real-time overlay representing the patient feature shown in the stereoscopic image; and
insert the real-time overlay into the left image data and the right image data to yield the stereoscopic image of the eye with the real-time overlay representing the patient feature at the z-location shown in the stereoscopic image.

7. The system of claim 1, the computer configured to:
generate a real-time overlay representing the patient feature shown in the stereoscopic image, the real-time overlay comprising a left real-time overlay and a right real-time overlay; and
insert the left real-time overlay into the left image data and the right real-time overlay into the right image data to yield the stereoscopic image of the eye with the real-time overlay representing the patient feature at the z-location shown in the stereoscopic image.

8. The system of claim 7, the computer configured to:
detect that the real-time overlay is aligned with the depth overlay; and
provide a notification that the real-time overlay is aligned with the depth overlay.

9. The system of claim 7, the computer configured to:
detect that the real-time overlay is aligned with the depth overlay; and
change one or more graphical features of the real-time overlay in response to detecting that the real-time overlay is aligned with the depth overlay.

10. The system of claim 7, the computer configured to:
detect that the real-time overlay is moving closer to alignment with the depth overlay; and
change one or more graphical features of the real-time overlay in response to detecting that the real-time overlay is moving closer to alignment with the depth overlay.

11. The system of claim 1, the computer configured to:
perform image processing to identify the patient feature shown in the stereoscopic image of the eye; and
calculate an adjustment to a relative distance between the eye and the system to align the patient feature with the depth overlay.

12. The system of claim 11, the computer configured to:
provide a description of the adjustment to a user.

13. The system of claim 11, the computer configured to:
detect that the adjustment has been performed to align the patient feature with the depth overlay; and
provide a notification that the patient feature is aligned with the depth overlay.

14. The system of claim 11, the computer configured to:
automatically perform the adjustment to align the patient feature with the depth overlay.

15. The system of claim 1, the computer configured to:
perform image processing to identify the patient feature shown in the stereoscopic image of the eye;
detect that the patient feature is not aligned with the depth overlay; and
provide a notification that the eye is not at the z-location.

16. The system of claim 1, further comprising a display device configured to present the stereoscopic image to a viewer by:
presenting the left image data to a left eye of the viewer; and
presenting the right image data to a right eye of the viewer.

17. The system of claim 16, the display device comprises oculars configured to:
present the left image data to a left ocular of the oculars; and
present the right image data to a right ocular of the oculars.

18. The system of claim 16, the display device comprises a headset configured to:
present the left image data to a left screen of the headset; and
present the right image data to a right screen of the headset.

19. The system of claim 16, the display device comprises a display configured to:
present the left image data polarized for the left eye of the viewer; and
present the right image data polarized for the right eye of the viewer.

20. The system of claim 16, the display device comprises a display configured to:
present the left image data angled for the left eye of the viewer; and
present the right image data angled for the right eye of the viewer.

21. The system of claim 1, wherein:
the patient feature comprises a pupil of the eye; and
the depth overlay is substantially a same size as the pupil.

22. The system of claim 1, wherein:
the patient feature comprises a limbus of the eye; and
the depth overlay is substantially a same size as the limbus.

23. The system of claim 1, wherein:
the patient feature comprises an eyeball of the eye; and
the depth overlay is substantially a same size as the eyeball.

24. The system of claim 1, the computer configured to:
detect that the patient feature is aligned with the depth overlay; and
provide a notification that the patient feature is aligned with the depth overlay.

25. The system of claim 1, the computer configured to:
detect that the patient feature is aligned with the depth overlay; and
change one or more graphical features of the depth overlay in response to detecting that the patient feature is aligned with the depth overlay.

26. The system of claim 1, the computer configured to:
detect that the patient feature is moving closer to alignment with the depth overlay; and
change one or more graphical features of the depth overlay in response to detecting that the patient feature is moving closer to alignment with the depth overlay.

27. A method for tracking and imaging an eye of a patient, comprising:
tracking a patient feature of the patient;
generating left image data and right image data of the eye and the patient feature to yield a stereoscopic image of the eye and the patient feature;
generating, by a computer, a depth overlay representing the patient feature at a z-location relative to a z-axis of a system coordinate system, the depth overlay comprising a left depth overlay and a right depth overlay; and
inserting, by the computer, the left depth overlay into the left image data and the right depth overlay into right image data to yield the stereoscopic image of the eye with the depth overlay representing the patient feature at the z-location.

28. The method of claim 27, wherein:
the patient feature comprises a pupil of the eye; and
the computer is configured to determine the z-location of the depth overlay according to an anterior chamber depth.

29. The method of claim 27, further comprising:
generating a real-time overlay representing the patient feature shown in the stereoscopic image; and
inserting the real-time overlay into the left image data and the right image data to yield the stereoscopic image of the eye with the real-time overlay representing the patient feature at the z-location shown in the stereoscopic image.
